(19) United States
(12) Patent Application Publication
(10) Pub. No.: US 2026/0306552 A1
(43) Pub. Date: Oct. 8, 2026

(54) METHODS OF PREVENTING ON-TARGET GENOTOXICITY INDUCED BY NUCLEASES

(71) Applicants: INSTITUT NATIONAL DE LA SANTÉ ET DE LA RECHERCHE MÉDICALE, Paris (FR); CHU DE BORDEAUX, Talence (FR); UNIVERSITÉ DE BORDEAUX, Bordeaux (FR)

(72) Inventors: François MOREAU-GAUDRY, Bordeaux (FR); Aurélie BEDEL, Bordeaux (FR); Sabrina FAYET, Bordeaux (FR)

(21) Appl. No.: 19/483,422

(22) PCT Filed: May 10, 2024
(86) PCT No.: PCT/EP2024/062916

§ 371 Date: Nov. 11, 2025

(30) Foreign Application Priority Data
May 12, 2023 (EP) 23305760.3

(51) Int. Cl. A61K 35/545; C12N 9/22; C12N 15/11; C12N 15/90

(52) U.S. Cl. CPC A61K 35/545; C12N 9/226; C12N 15/11; C12N 15/907; C12N 2310/20

(57) ABSTRACT

The CRISPR-Cas9 system has revolutionized our ability to precisely modify the genome and has impelled gene editing into clinical applications. Comprehensive analysis of gene editing products at the targeted cut-site revealed a complex spectrum of outcomes. ON-target genotoxicity is underestimated with standard PCR-based methods and necessitates appropriate and sensitive detection methods. Here, the inventors developed two complementary Fluorescence-Assisted Megabase-scale Rearrangements Detection (FAMReD) systems that enable detection, quantification, and cell sorting of edited cells with megabase-scale loss of heterozygosity (LOH). They revealed rare complex chromosomal rearrangements caused by Cas9-nuclease and showed that LOH frequency depends on cell division rate during editing and p53 status. Cell cycle arrest during editing suppressed the appearance of LOH without compro-2024/235844 mising editing. These data were confirmed in human stem/progenitor cells, suggesting that clinical trials should consider p53 status and cell proliferation rate during editing to limit this risk and design safer protocols.

### FIELD OF THE INVENTION

The present invention is in the field of medicine, in particular, gene therapy.

### BACKGROUND OF THE INVENTION

Targeted nucleases, and in particular the CRISPR-Cas9 system, are a breakthrough that has propelled gene therapy into a new era¹⁻⁴. Important advances are illustrated by several ongoing preclinical and clinical studies in fields such as immunotherapy, virology, and monogenic diseases. Nevertheless, a major concern is the potential genotoxicity of DNA double-strand breaks (DSB), which arise from incorrect or ineffective DNA repair and DNA damage response. The risk of genomic instability seems to be the Achilles heel of CRISPR-Cas9. Detailed genotyping of edited cells revealed that the full spectrum of Cas9-induced outcomes might be more complex than the sole induction of insertion and deletions InDels at the targeted locus (ON-target genotoxicity)⁵. Kilobase-scale deletions, inversions and insertions have been reported in primary murine and human cells⁶⁻¹⁰. Chromosome truncations associated with megabasic loss-of-heterozygosity with copy losses (CL-LOH) have been reported not only in cancer cell lines^{11,12} but also in p53-proficient human induced pluripotent stem cells (hiPSC) and embryos^{9,13}. Terminal megabase-scale copy-neutral LOHs (CN-LOHs) without loss of genetic material have also been observed in cancer cell lines¹⁴, human embryonic stem cells (hESC) and hiPSC⁹⁻¹⁵. Gene editing of human hematopoietic stem/progenitor cells (HSPC) at the beta-globin cluster also gave rise to CN-LOHs, associated with an abnormal methylation profile of 11p15 imprinting centers (duplication of maternal or paternal allele) with altered transcriptional activity.¹⁶ Finally, chromothripsis¹⁷ and entire chromosome losses^{13,18-21} can occur in response to a single DSB. Understanding the mechanisms involved in ON-target genotoxicity is a first step to limit them and improve the safety of CRISPR-based clinical trials.

### SUMMARY OF THE INVENTION:

The present invention is defined by the claims. In particular, the present invention relates to methods of preventing the ON-target genotoxicity induced by a nuclease.

### DETAILED DESCRIPTION OF THE INVENTION

The CRISPR-Cas9 system has revolutionized our ability to precisely modify the genome and has impelled gene editing into clinical applications. Comprehensive analysis of gene editing products at the targeted cut-site revealed a complex spectrum of outcomes. ON-target genotoxicity is underestimated with standard PCR-based methods and necessitates appropriate and sensitive detection methods. Here, the inventors developed two complementary Fluorescence-Assisted Megabase-scale Rearrangements Detection (FAMReD) systems that enable detection, quantification, and cell sorting of edited cells with megabase-scale loss of heterozygosity (LOH). They revealed rare complex chromosomal rearrangements caused by Cas9-nuclease and showed that LOH frequency depends on cell division rate during editing and p53 status. Cell cycle arrest during editing suppressed the appearance of LOH without compromising editing. These data were confirmed in human stem/progenitor cells, suggesting that clinical trials should consider p53 status and cell proliferation rate during editing to limit this risk and design safer protocols.

Accordingly, the first object of the present invention relates to a method of preventing the ON-target genotoxicity induced by a nuclease in population of cells comprising the steps of i) inducing the G0/G1 phase cell cycle arrest in the population of cells and ii) editing the population of cells of step i) set with the nuclease.

As used herein, the term “genotoxicity” has its general meaning in the art and refers to a damage to the genetic material of a live cell. The damage to the genetic material of a cell may include, for example, damage resulting from nucleotide or polynucleotide deletion, addition, point mutation, dimerization or recombination, and DNA breakage or degradation. The damage may also include that which is evidenced by chromosomal numerical abnormalities, such as polyploidy or aneuploidy. In particular, the term “genotoxicity” describes a deleterious action of an agent (e.g. a nuclease) on a cell's genetic material affecting its integrity. Said genotoxicity can be potentially mutagenic or carcinogenic, specifically those capable of causing genetic mutation and of contributing to the development of tumors.

As used herein, the term “ON-target genotoxicity” refers to the presence of unwanted genomic modifications at the targeted locus by the nuclease. ON-target genotoxicity includes insertion/deletion of a few bases to megabase-scale rearrangements (“ON-target megabase-scale genotoxicity”). In particular, the method of the present invention is particularly suitable for preventing ON-target megabase-scale genotoxicity. More particularly, the method of the present invention is particularly suitable for reducing LOH frequency. As used herein, the term “loss of heterozygosity” or “LOH” has its general meaning in the art and refers to a type of genetic abnormality in diploid organisms in which one copy of an entire gene and its surrounding chromosomal region are lost. In particular, the method of the present invention is particularly suitable for reducing the nuclease-induced megabase-scale LOHs that include megabase-scale loss-of-heterozygosity with copy losses (CL-LOH) and megabase-scale copy-neutral loss-of-heterozygosity (CN-LOH).

As used herein, the term “reduce” or “prevent” or grammatical variations thereof means, respectively, lessening the effects or keeping the effects from occurring completely. In particular, the term “reduce” means a reduction by a statically significant amount, for example, an increase of at least 10%, or at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “editing” refers to a type of genetic engineering in which a nucleic acid sequence is inserted, replaced, or removed from a target nucleic acid sequence, e.g., the genome of a cell, using one or more nucleases according to the present invention. The nucleases create specific double-strand breaks (DSBs) at desired locations in the genome, and harness the cell's endogenous mechanisms to repair the induced break by homology-directed repair (HDR) (e.g., homologous recombination). Thus the term “editing” refers to a method of altering a nucleic acid sequence of a polynucleotide (e.g., a naturally-occurring wild type nucleic acid sequence or a naturally-occurring mutated nucleic acid sequence by introducing a change to a specific genomic target; the genomic target may include a chromosomal region, a coding polynucleotide (e.g., a gene), a promotor, a non-coding polynucleotide, or any nucleic acid sequence. The changes to a nucleic acid may include deletion, addition and other changes to the nucleic acid sequence in the genome.

As used herein, the “target nucleic acid sequence,” “target sequence” or “target region” means a specific sequence or the complement thereof that one wishes to alter.

As used herein, the term “nuclease” includes an enzyme that induces a break in a nucleic acid sequence, e.g., a single or a double strand break in a double-stranded DNA sequence.

In some embodiments, the nuclease is a TALEN. As used herein, the term “TALEN” has its general meaning in the art and refers to a transcription activator-like effector nuclease, an artificial nuclease which can be used to edit a target gene. TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29: 149-153). By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29: 135-6; and Boch et al. (2009) Science 326: 1509-12; Moscou et al. (2009) Science 326:3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29: 149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29: 143-8; Hockemeyer et al. (2011) Nature Biotech. 29:731-734; Wood et al. (2011) Science 333: 307; Doyon et al. (2010) Nature Methods 8:74-79; Szczepek et al. (2007) Nature Biotech. 25: 786-793; and Guo et al. (2010) J. Mol. Biol. 200: 96). The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (Miller et al. (2011) Nature Biotech. 29:143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17: 11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

In some embodiments, the nuclease of the present invention is a Zinc-Finger Nuclease (ZFN). As used herein, “Zinc Finger Nuclease” or “ZFN” has its general meaning in the art and refers to a zinc finger nuclease, an artificial nuclease which can be used to edit a target gene. Like a TALEN, a ZFN comprises a DNA-modifying domain, e.g., a nuclease domain, e.g., a FokI nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188: 773-782; and Kim et al. (1996) Proc. Natl. Acad. Sci. USA 93: 1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95: 10570-5). Also like a TALEN, a ZFN can create a DSB in the DNA, which can create a frame-shift mutation if improperly repaired, e.g., via non-homologous end joining, leading to a decrease in the expression of a target gene in a cell.

In some embodiments, the nuclease is a CRISPR/Cas nuclease. As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpfl belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG for S. Pyogenes Cas9) protospacer adjacent motif (PAM) to specify the cut site (the 3^{rd} or the 4^{th} nucleotide upstream from PAM).

In some embodiments, the CRISPR/Cas nuclease is the Cas9. As used herein, the term “Cas9” or “Cas9 endonuclease” (also known as COG3513, Csxl2, Cas5, or Csnl) refers to a CRISPR-associated protein with two nuclease domains that uses a crRNA: tracRNA duplex for site-specific double-stranded cleavage of DNA. According to the present invention, the terms may refer to a wild-type Cas9 protein, or any variant, including mutants, homologs, orthologs, that mediate RNA-guided double-stranded or single-stranded cleavage of DNA. In particular, the term “Cas9” refers wild type Cas9 from Streptococcus pyogenes (NCBI Reference Sequence: NC_017053.1), Corynebacterium ulcerans (NCBI Refs: NC_015683.1,NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

In particular, the nuclease involves the use of one or more guide RNA(s). As used herein, the term “guide RNA” or “gRNA” has its general meaning in the art and is a particular type of guide nucleic acid which associates with a CRISPR/Cas nuclease (e.g. Cas9), directing the nuclease to a specific sequence in a DNA molecule that includes complementarity to protospacer sequence of the guide RNA.

Thus, in some embodiments, a further object of the present invention relates to a method of altering a target sequence of a nucleic acid molecule in a population of cells for which a G0/G1 phase cell cycle arrest is induced that comprises the step consisting in contacting the target nucleic acid sequence of said population of cells with (a) the nuclease and (b) one or more guide RNA(s).

In some embodiments, the method of the present invention is used to alter a target polynucleotide sequence of interest in the population of cells for any purpose.

The population of cells can be a population of cells isolated from any multicellular organism, e.g., a plant cell (e.g., a rice cell, a wheat cell, a tomato cell, an Arabidopsis thaliana cell, a Zea mays cell, and the like), a cell from a multicellular protist, a cell from a multicellular fungus, an animal cell such as a cell from an invertebrate animal (e.g., fruit fly, cnidarian, echinoderm, nematode, etc.) or a cell from a vertebrate animal (e.g., fish, amphibian, reptile, bird, mammal, etc.), a cell from a human, a cell from a healthy human, a cell from a human patient, a cell from a cancer patient, etc. In some cases, the cell with induced gene regulation can be transplanted to a subject (e.g., patient). For instance, the cell can be derived from the subject (e.g., patient) to be treated.

In some embodiments, the cell is an eukaryotic cell. Any type of eukaryotic cell may be of interest, such as a stem cell, e.g., embryonic stem cell, induced pluripotent stem cell, adult stem cell (e.g., mesenchymal stem cell, neural stem cell, hematopoietic stem cell, organ stem cell), a progenitor cell, a somatic cell (e.g., fibroblast, hepatocyte, heart cell, liver cell, pancreatic cell, muscle cell, skin cell, blood cell, neural cell, immune cell), and any other cell of the body, e.g., human body. The cells can be primary cells or eukaryotic cell cultures derived from a subject, e.g., an animal subject or a human subject, and allowed to grow in vitro for a limited number of passages. In some embodiments, the cells are disease cells or derived from a subject with a disease. For instance, the cells can be cancer or tumor cells.

In some embodiments, the eukaryotic cell is selected from the group consisting of hematopoietic progenitor cells, hematopoietic stem cells (HSCs), pluripotent cells (i.e. embryonic stem cells(ES) and induced pluripotent stem cells (iPS)). More preferably the eukaryotic cell is a hematopoietic stem cell.

In some embodiments, the cells are p53-proficient cells or p53-deficient cells. As used herein, the term “p53” has its general meaning in the art and refers the tumor suppressor protein containing transcriptional activation, DNA binding, and oligomerization domains. The p53 protein responds to diverse cellular stresses to regulate expression of target genes, thereby inducing cell cycle arrest, apoptosis, senescence, DNA repair, or changes in metabolism. As used herein, the term “p53-proficient cell” refers to a cell has a “wild type TP53 gene” i.e. a gene encoding p53 protein which does not harbor a p53 dominant negative mutation. Conversely, p53-deficient cells are typically characterized by the presence of at least one p53 dominant negative mutation. As used the term “p53 dominant negative mutation” has its general meaning in the art and refers to any mutation which results in to a dysfunction of the protein leading to the loss of its transcriptional activity associated with a negative effect on the wild type protein in heterozygous status. p53 loss of function mutations have fully been exemplified in the prior art and thus the skilled man in the art can easily identifies p53 dominant negative mutations (Petitjean A, Mathe E, Kato S, Ishioka C, Tavtigian S V, Hainaut P, Olivier M. Impact of mutant p53 functional properties on TP53 mutation patterns and tumor phenotype: lessons from recent developments in the IARC TP53 database. Hum Mutat. 2007 Jun; 28(6): 622-9) (http://p53.iarc.fr/). p53 dominant negative mutations include somatic and germline mutations indentified in previous studies are mainly missense mutations. Examples of p53 dominant negative mutations include but are not limited to p.R175H, p.R248W and p.R273H.

In some embodiments, the target polynucleotide sequence of interest in the eukaryotic cell is used to generate a mutate cell, which results in a genotype that differs from its original genotype. In some embodiments, the target polynucleotide sequence of interest in the eukaryotic cell is altered to correct or repair a genetic mutation (e.g., to restore a normal phenotype to the cell). In some embodiments, the target polynucleotide sequence of interest in the eukaryotic cell is altered to induce a genetic mutation (e.g., to disrupt the function of a gene or genomic element). In some embodiments, the alteration may be a homozygous alteration or a heterozygous alternation. In some embodiments, the alteration may be an insertion, deletion, or the combination thereof. As will be appreciated by those skilled in the art, an insertion/deletion in a coding region of a genomic sequence will result in a frameshift mutation or a premature stop codon. In some embodiments, the alteration may be a point mutation. In some embodiments, the alteration consists in introducing a plurality of point mutations.

In some embodiments, the method of the present invention of the present invention is used to generate a knock-out of a target polynucleotide sequence. The knocking out of a selected polynucleotide sequence can be useful for many applications, such as knocking out a target polynucleotide sequence of interest in the eukaryotic cell clone in vitro for research purposes; and knocking out a target polynucleotide sequence ex vivo for treating or preventing a disorder associated with increased expression of the target polynucleotide sequence. As used herein, the term “knock out” includes deleting all or a portion of the target polynucleotide sequence in a way that mutes the function of the target polynucleotide sequence.

In some embodiments, the alternation may result in a change of the target polynucleotide sequence of interest from an undesired sequence to a desired sequence. In some embodiments, the method of the present invention is used to correct any type of mutation or error in a target polynucleotide sequence of interest, including but not limited to inserting a nucleotide sequence that is missing from a target polynucleotide sequence due to a deletion, deleting a nucleotide sequence from a target polynucleotide sequence due to an insertion mutation, and replacing an incorrect nucleotide sequence with a correct nucleotide sequence.

In some embodiments, the alteration results in reduced or increased expression of a target polynucleotide sequence of interest.

As used herein, the term “cell cycle” has its general meaning and refers to the biological process through which cells replicate and make two new cells. Cell cycle has different stages called G0, G1, S, G2, and M. Resting cells that are not dividing are in a phase called G0. During division, the cell undergoes 4 distinct stages termed G1, S, G2, and M (mitosis). These periods of activity are separated by regulatory checkpoints at major transition phases. These checkpoints include G0/G1 which regulates the entry of a quiescent cell back into the cycle, G1/S, and G2/M. As used herein, the term “G0/G1 phase cell cycle arrest” indicates that population of cells cannot undergo cell division and are blocked in the G0/G1 phase of the cell cycle. According to the present invention, the G0/G1 phase cell cycle arrest is induced by any agent that selectively induces G1 cell cycle arrest. Thus, when treated with said agent, the percentage of cells in the G1 phase increase, while the percentage of cells in the G2/M phase and S phase decrease. In some embodiments, the agent is a compound that induces substantially pure (i.e., “clean”) G1 cell cycle arrest in the population of cells (e.g., wherein treatment with the agent induces cell cycle arrest such that the majority of cells are arrested in G1 as defined by standard methods (e.g., propidium iodide staining or others) and with the population of cells in the G2/M and S phases combined being 20%, 15%, 12%, 10%, 8%, 6%, 5%, 4%, 3%, 2%, 1% or less of the total cell population).

Typically,. As used herein, the term “selective CDK4/6 inhibitor compound” refers to a compound that selectively inhibits at least one of CDK4 and CDK6 or whose predominant mode of action is through inhibition of CDK4 and/or CDK6. As used herein, the term “cyclin-dependent kinase 4” or “CDK4” has its general meaning in the art and refers the cell division protein kinase 4 that is an enzyme that in humans is encoded by the CDK4 gene. As used herein, the term “cyclin-dependent kinase 6” or “CDK6” has its general meaning in the art and refers the cell division protein kinase 6 that is an enzyme that in humans is encoded by the CDK6 gene.

Preferably, selective CDK 4/6 inhibitors are compounds that generally have a lower 50% inhibitory concentration (IC50) for CDK4 and/or CDK6 than for other kinases. In some embodiments, the selective CDK4/6 inhibitor can have an IC50 for CDK4 or CDK6 that is at least 2, 3, 4, 5, 6, 7, 8, 9, or 10 times lower than the compound's IC 50s for other CDKs (e.g., CDK1 and CDK2). In some embodiments, the selective CDK4/6 inhibitor can have an IC50 for CDK 4 or CDK6 that is at least 20, 30, 40, 50, 60, 70, 80, 90, or 100 times lower than the compound's IC50s for other CDKs. In some embodiments, the selective CDK4/6 inhibitor can have an IC50 that is more than 100 times or more than 1000 times less than the compound's IC50s for other CDKs. In some embodiments, the selective CDK4/6 inhibitor compound is a compound that selectively inhibits both CDK4 and CDK6. In some embodiments, the CDK4/6 inhibitor is a poor inhibitor (e.g., >1 μM in vitro IC50) of one or more tyrosine kinases. In some embodiments, the CDK4/6 inhibitor is a high potency inhibitor of serine and/or theonine kinases. In some embodiments, the CDK4/6 inhibitor is a poor CDK1 inhibitor (e.g., (e.g., >1 μM in vitro IC50). In some embodiments, the CDK4/6 inhibitor is characterized by having a 10-fold or 50-fold or 100-fold or greater relative potency for inhibiting CDK4 or CDK6 as compared to CDK1.

Selective CDK4/6 inhibitors that can be used according to the presently disclosed methods include any known small molecule (e.g., <1000 Daltons, <750 Daltons, or less than <500 Daltons), selective CDK4/6 inhibitor, or pharmaceutically acceptable salt thereof. In some embodiments, the inhibitor is a non-naturally occurring compound (i.e., a compound not found in nature). Several classes of chemical compounds have been reported as having CDK4/6 inhibitory ability (e.g., in cell free assays).

Selective CDK4/6 inhibitors useful in the presently disclosed methods can include, but are not limited to, pyrido[2,3-d]pyrimidines (e.g., pyrido[2,3-d]pyrimidin-7-ones and 2-amino-6-cyano-pyrido[2,3-d]pyrimidin-4-ones), triaminopyrimidines, aryl[a]pyrrolo[3,4-d]carbazoles, nitrogen-containing heteroaryl-substituted ureas, 5-pyrimidinyl-2-aminothiazoles, benzothiadiazines, acridinethiones, and isoquinolones. In some embodiments, the pyrido[2,3-d]pyrimidine is a pyrido[2,3-d]pyrimidinone. In some embodiments the pyrido[2,3-d]pyrimidinone is pyrido[2,3-d]pyrimidin-7-one. In some embodiments, the pyrido[2,3-d]pyrimidin-7-one is substituted by an aminoaryl or aminoheteroaryl group. In some embodiments, the pyrido[2,3-d]pyrimidin-7-one is substituted by an aminopyridine group. In some embodiments, the pyrido[2,3-d]pyrimidin-7-one is a 2-(2-pyridinyl)amino pyrido[2,3-d]pyrimidin-7-one. For example, the pyrido[2,3-d]pyrimidin-7-one compound can have a structure of Formula (II) as described in U.S. Patent Publication No. 2007/0179118 to Barvian et al., herein incorporated by reference in its entirety. In some embodiments, the pyrido[2,3-d]pyrimidine compound is 6-acetyl-8-cyclopentyl-5-methyl-2-(5-piperazin-1-yl-pyridin-2-ylamino)-8H-pyrido-[2,3-d]pyrimidin-7-one (i.e., PD 0332991 or palbociclib) or a pharmaceutically acceptable salt thereof. See Toogood et al., J. Med. Chem., 2005, 48, 2388-2406. In some embodiments, the pyrido[2,3-d]pyrimidinone is a 2-amino-6-cyano-pyrido[2,3-d]pyrimidin-4-ones. Selective CDK4/6 inhibitors comprising a 2-amino-6-cyano-pyrido[2,3-d]pyrimidin-4-one are described, for example, by Tu et al. See Tu et al., Bioorg. Med. Chem. Lett., 2006, 16, 3578-3581. Suitable 5-pyrimidinyl-2-aminothiazole CDK4/6 inhibitors are described by Shimamura et al. See Shimamura et al., Bioorg. Med. Chem. Lett., 2006, 16, 3751-3754. Useful benzothiadiazine and acridinethiones compounds include those, for example, disclosed by Kubo et al. See Kubo et al., Clin. Cancer Res. 1999, 5, 4279-4286 and in U.S. Patent Publication No. 2004/0006074, herein incorporated by reference in their entirety. In some embodiments, the benzothiadiazine is substituted by one or more halo, haloaryl, or alkyl group. In some embodiments, the benzothiadiazine is selected from the group consisting of 4-(4-fluorobenzylamino)-1,2,3-benzothiadiazine-1,1-dioxide, 3-chloro-4-methyl-4H-benzo[e][1,2,4]thiadiazine-1,1-dioxide, and 3-chloro-4-ethyl-4H-benzo[e][1,2,4]thiadiazine-1,1-dioxide. In some embodiments, the acridinethione is substituted by one or more amino or alkoxy group. In some embodiments, the acridinethione is selected from the group consisting of 3-amino-10H-acridone-9-thione (3ATA), 9(10H)-acridinethione, 1,4-dimethoxy-10H-acridine-9-thione, and 2,2′-diphenyldiamine-bis-[N,N′-[3-amido-N-methylamino)-10H-acridine-9-thione]].

In particular, CDK4/6 inhibitors that may be useful in the methods of the present invention include abemaciclib, palbociclib, and ribociclib. Abemaciclib (N-[5-[(4-ethylpiperazin-1-yl)methyl]pyridin-2-yl]-5-fluoro-4-(7-fiuoro-2-methyl-3-propan-2-ylbenzimidazol-5-yl)pyrimidin-2-amine) is described in U.S. Pat. No. 7,855,211 incorporated herein. Palbociclib (6-Acetyl-8-cyclopentyl-5-methyl-2-{[5-(1-piperazinyl)-2-pyridinyl]amino}pyrido[2,3-d]pyrimidin-7(8H)-one) is described in U.S. Pat. No. 7,208,489 incorporated herein. Ribociclib (7-cyclopentyl-N,N-dimethyl-2-[(5-piperazin-1-ylpyridin-2-yl)amino]pyrrolo[2,3-d]pyrimidine-6-carboxamide) is described in U.S. Pat. Application No. 2010/0105653 incorporated herein.

In some embodiments, the editing of the population of cells and the G0/G1 phase cell cycle arrest can occur simultaneously. In particular, the agent directly and selectively induces the G0/G1 cell cycle arrest in cells, without the need for prolonged (e.g., 48 hour or longer) treatment with the agent prior to exposure with the nuclease.

The methods described herein can be used in ex vivo therapy. Ex vivo therapy can comprise administering a composition (e.g., a cell) generated or modified outside of an organism to a subject (e.g., patient). In some embodiments, the composition (e.g., a cell) can be generated or modified by the methods disclosed herein. For example, ex vivo therapy can comprise administering a cell generated or modified outside of an organism to a subject (e.g., patient), wherein the primary cell has been cultured in vitro in accordance with the methods of the present invention. In some embodiments, the composition (e.g., a cell) can be derived from the subject (e.g., patient) to be treated by ex vivo therapy. In some embodiments, ex vivo therapy can include cell-based therapy, such as adoptive immunotherapy.

Thus, a further object of the present invention relates to a method of therapy in a patient in need thereof, the method comprising transplanting a therapeutically effective amount of a population of edited eukaryotic cells obtained by the methods herein disclosed.

The methods described herein can also be used in in vivo therapy. Thus, a further object of the present invention relates to a method of therapy in a patient in need thereof, comprising administering to the patient a therapeutically effective amount of the nuclease in combination with a therapeutically effective amount of the agent that induces the G0/G1 cell cycle arrest.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is the agent that induces the G0/G1 cell cycle arrest and wherein the other drug is the nuclease.

As used herein, the term “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

Typically, the drugs of the present invention are administered to the subject in the form of a pharmaceutical composition which comprises a pharmaceutically acceptable carrier.

The present invention further provides kits containing reagents for performing the above-described methods, including all component for performing the edition as described herein. To that end, one or more of the reaction components, e.g. the nucleases and the agent that induces the G0/G1 cell cycle arrest for the methods disclosed herein can be supplied in the form of a kit for use. In some embodiments, the kit comprises (a) at least one nuclease or a polynucleotide encoding thereof, and (b) one or more guide RNA molecules designed for guiding the nuclease(s) and (c) and one agent that induces the G0/G1 cell cycle arrest. In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the guide RNA or nucleases or verifying the target nucleic acid's status, and buffers or culture media for the reactions. The kit may also include one or more of the following components: supports, terminating, modifying or digestion reagents, osmolytes, and an apparatus for detection. The components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0 ° C., preferably at or below- 20° C., or otherwise in a frozen state. The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### FIGURES

FIG. 1. Cell division affects Cas9-induced LOH frequency. Fluorescent cells correspond to cells with LOH induced by CRISPR-Cas9. Cells (TP53-WT and TP53^{-/-}) were exposed to palbociclib to synchronize cells in G0/G1 phase before and during editing. Cell cycle analysis was performed by flow cytometry to confirm synchronization. Fluorescent cell quantification after Cas9: SORCS1 editing (results normalized on fluorescent cell rate in non-synchronized cells, n≥3 independent experiments, mean±SD). InDels were quantified by sequencing and ICE. P: Palbociclib.

FIG. 2. Low cell proliferation mitigates CRISPR-induced LOH frequency. a, hFFs edited and stained with a fluorescent cell tracker. 48 h after editing, the 10% of cells with the highest MFI (“low-divided” cells) and the 10% of cells with the lowest MFI (“most divided” cells) were sorted by FACS. b, After day 14, fluorescent cells revealing LOH are analyzed by flow cytometry in each cell fraction (mean±SD, n=3) . c, InDels were quantified by sequencing and ICE analysis in each cell fraction (n=3 independent experiments, mean±SD). Anova test used to compare multiple groups, Mann-Whitney or t-test to compare two groups.

FIG. 3. Low cell division rate prevents LOH targeting b-globin region in hCD34⁺ cells. a, Left Schematic representation of globin region targeting in hCD34⁺ cells from cord blood, and H19/IGF2/CDKNIC imprinting center in chr11p. Right, Identification of three SNPs by allele-specific qPCR in H19/IGF2/KCNQI imprinting center, telomeric to globin region in Chr11p. Representative curves of heterozygous profile (SNP, two curves before editing in parental hCD34⁺ cells) and loss of SNP (one curve, after editing in the event of LOH (lower panel). b, Edited hCD34⁺ cells stained with a fluorescent cell-tracker (red dye). 48 h after, the 10% highest MFI cells (low division rate) and the 10% lowest MFI cells (high division rate) were sorted by FACS and seeded in methylcellulose. Colonies were picked up after 2-3 weeks for SNP analysis by allele-specific qPCR. Percentage of clones with LOH (at least 2 SNPs loss) in low-divided clones (more clonogenic cells, n=500) and in high-divided clones (less clonogenic cells, n=82 clones). Chi-square test used to compare LOH frequencies.

FIG. 4. Synchronization of hCD34+ cells from cord blood by Palbociclib exposure during 48 hours prevents the formation of micronuclei (small DNA-containing structures isolated from the main nucleus evoking a massive DNA rearrangement called chromothripsis).
a, Effect of palbociclib on the HSPCs cell cycle. At day 0 at day 2 without or with 1 μmol palbociclib and at day 7 demonstrating the efficient blockade of CD34⁺ cells in GOG1 cells.
b, Effect of palbociclib on micronuclei frequency measured by flow cytometry (In Vitro MicroFlow®). Control without transfection or 48 hours post transfection targeting HBG1/HBG2 human promoters without or with 1 μmol palbociclib exposure.

### METHODS

### Cell culture

Human foreskin fibroblasts immortalized with hTERT (hFF hTERT) used in hFAMReD system were from ATCC® (CRL 4001, BJ-5ta). They were partially invalidated for UROS and totally invalidated for TP53 using an RNP made of Cas9 protein complexed with a gRNA targeting UROS exon 4 and TP53 exon 4, respectively. hFFs were maintained in Dulbecco's modified Eagle's medium (DMEM with glucose (4.5 g. L⁻¹), L-Glutamine (1 g. L⁻¹) and pyruvate supplemented with 20% fetal bovine serum, MEM non-essential amino acids 100X (Gibco® by ThermoFisher scientific, Carlsabad, CA, USA), 100 U/mL penicillin, and 100 μg/mL streptomycin (all from Eurobio, Courtaboeuf, France).

Human CD34⁺ HSPCs were isolated from the cord blood of healthy donors from Bagatelle Hospital, according to the ethical institutional review board of Bagatelle Hospital, (Maison de Santé Protestante de Bordeaux, Talence, France) and with the mother's informed consent. Briefly, mononuclear cells were isolated by Ficoll gradient. CD34⁺ cells were purified according to the manufacturer's instructions (Human CD34-Positive Selection kit II ref 17865 from Stem Cell Technologies) and purity was analyzed by flow cytometry using PE-conjugated anti-CD34 antibody (clone 561 Biolegend (San Diego CA, USA) 25 μg/mL, lot #B2044487, 5 μL/test). Cryopreserved CD34⁺ cells were thawed and cultured in expansion medium consisting in Stem Span SFEM (Stem Cell Technologies) supplemented with hFlt3-L (50 ng/ml), SCF (50 ng/ml), human TPO (50 ng/ml), human IL3 (20 ng/ml) and human IL6 (10 ng/ml) (all from Peprotech), StemRegenin 1 (SR1) (1 μM) (Stem Cell Technologies), VPA valproic acid (500 μM) (Sigma-Aldrich), 100 U/mL penicillin, and 100 μg/mL streptomycin (Eurobio). Two days after thawing, CD34⁺ cells were transfected with Cas9 RNP (see below). After cell tracking staining and sorting, CD34⁺ cells were plated in 35-mm tissue culture dishes at 50 and 1000 cells/mL with 1 mL of methylcellulose medium (Stemcell Technologies, MethoCult H4034 Optimum). After 3 weeks, individual colonies were subsequently picked from plates and washed in PBS to remove all the methylcellulose. Cells were digested with proteinase K in lysis buffer (10 mmol/L Tris-Cl, pH 8.0, 50 mmol/L KCl, 2.5 mmol/L MgC12, 0.5% Tween 20, 100 mg/mL proteinase K) at 56° C. for 1 h, followed by a 10-min exposure at 95° C.

All cells were cultured in a standard humidified 37° C., 5% CO₂ incubator.

### Transfection and Gene Editing Tools

Cells were transfected by electroporation using the AMAXA™ 4D-Nucleofector™ device (Lonza®, Bale, Switzerland) with P3 Primary Cell Line and CZ-167 and D0-100 programs for fibroblasts (murine and human) and hCD34⁺ cells, respectively. In brief, 200,000 cells were nucleofected with 16.9 μg Cas9 RNP and 5 μM of Alt-R® Cas9 Electroporation Enhancer. To form RNP, Alt-R® S.p. Cas9 Nuclease V3 protein (or either HiFi Cas9, Cas9^{D10A} or dCas9 when specified) was complexed to crRNA: tracrRNA according to the manufacturer's instructions. Then complexes were incubated for 20 min at room temperature before electroporation. Cas9 proteins and crRNA were purchased from Integrated DNA Technologies, Coralville, USA.

### Indels Quantification

Genomic DNA of edited cells, and their associated controls, was extracted using Nucleospin® Tissue (Macherey-Nagel®) according to the manufacturer's protocol. The genomic region flanking the expected cut-site was amplified by PCR (HotStarTaq Plus DNA polymerase, Qiagen®, Venlo, Netherlands) with adequate primers (Supplementary Table 3). PCR products were purified with Nucleospin® Gel and PCR Clean-up (Macherey-Nagel). Sanger sequencing was done on purified PCR products and sequenced by LIGHTRUN (GATC Biotech, Konstanz, Germany). Sanger sequencing data were analyzed using ICE v2 CRISPR Analysis tool (ICE) software (Synthego, Redwood City, USA). Purified PCR products from non-edited cells were used as control chromatogram.

### Cell Cycle Analysis and Synchronization

Cell cycle analyses were performed to check synchronization efficiency. Briefly, cells either synchronized or not were harvested and washed twice with PBS, then fixed with 70% ethanol in PBS overnight at 4° C. Cells were washed twice with PBS and incubated with a mix containing RNAse (1 mg/ml) and PBS-propidium iodide (0.5μg/ml, for 15 min), both from Sigma-Aldrich, Saint Louis, USA. The samples were examined on a BD Biociences Accuri C6Plus apparatus and the data were analyzed with BD CSampler™ software (BD Biosciences, Le Pont de Claix, France).

To evaluate the impact of synchronization on the occurrence of LOH, cells were synchronized in G0/G1 phase by incubation with palbociclib, also named PF-00080665 or PD 0332991 (1 μM, Sigma Aldrich) 24 h before and 36 h-48 h after RNP transfection (supplementary FIG. 6). To synchronize cells in the G2/M phase, cells were incubated with RO-3306 (10 μM, Sigma Aldrich) for 24 h before RNP transfection and 24 after RNP transfection.

### Proliferation Assays

Cell tracking. Edited cells (hFFs or hCD34⁺ cells) were stained immediately after editing. To do so, cells were plated and stained for 3 hours with 5 μL of Cell Tracking Red Dye Kit (Abcam, reference ab 269446, Cambridge, UK) per 100 μl of medium. After 3 hours of incubation in the staining medium, cells were PBS-washed three times by centrifugation at 500 g for 10 min. After 48 hours, cells were FACS-sorted to isolate 10% cell tracking^{high} and 10% cell tracking^{low}cells and expanded (FACS Aria, BD). Comparison of initial and 48 h cell tracking curves was performed using FlowJo Software (BD biosciences).

### hFAMReD Fluorescent Cell Quantification and Sorting

At day 15 post-editing, 0.3 mM of 5-ALA were added to hFF media. After 16 h of exposure (overnight), cells were washed twice with 1X PBS and put back in fresh media. Upon LOH, loss of UROS can be detected by the appearance of fluorescence due to porphyrin accumulation. Following 8 hours of clearance, fluorescent cells were quantified by flow cytometry (data not shown). UV-sensitive porphyrins were excited at 488 nm and the emitted wavelength was approximately 667 nm, detected by the PE-Cy5A PMT channel (FACS Accuri, BD, Franklin Lakes, NJ, USA). FL-1 is a control green fluorescent channel used to exclude auto-fluorescent cells. Fluorescent-positive or -negative fractions were sorted by BD FACS Aria®.

### SNP Analysis by Sanger Sequencing of DOCK1/MGMT/UROS and by Allele-Specific Quantitative PCR in 11p15 Region

We used dbSNP (NCBI) to screen frequent SNPs in parental cells. In fluorescent hFFs, we tested SNPs in UROS, DOCK1 and MGMT by Sanger sequencing to confirm Chr10q LOH. The genomic regions flanking SNPs were amplified by PCR (HotStarTaq Plus DNA polymerase, Qiagen®, Venlo, Netherlands) with adequate primers (data not shown). PCR products were purified with Nucleospin® Gel and PCR Clean-up (Macherey-Nagel) and sequenced.

For the methylcellulose CFC assay, we selected and tested SNPs in H19, IGF2 and KCNQ1 to screen Chr11p LOH. SNP genotyping was performed by real-time quantitative PCR analysis (CFX Connect device, Biorad®) of genomic DNA with a common reverse primer and two SNP allele-specific forward primers. Only curves with Ct<37 were included for the analysis. To be considered as an SNP loss (homozygous), the profile can be only one or two curves with a delta Ct>6.

### Array CGH and SNP Array

Array CGH was performed on 8×60 k oligonucleotide microarrays (Agilent Technologies, CA). DNA was labeled (cyanine 3 or cyanine 5) using the Genomic DNA ULS Labeling Kit from Agilent Technologies and hybridized onto the microarrays according to the manufacturer's instructions (Agilent). Scanning of the microarrays was performed using a G5761A scanner (Agilent). Data analysis was carried out with Agilent Technologies software, namely Feature Extraction for Cytogenomics V5.0 to calculate the fluorescence ratio and Agilent CytoGenomics 5.2 to visualize chromosomal imbalances. Deletions and duplications in the heterozygous state were characterized by values of the log2 ratio of fluorescence intensities (cyanine 5/cyanine3) below −0.5 and above +0.3, respectively, with the statistical algorithm ADM2 used at a threshold of 5.

Combined SNP/CGH array was performed on Genetisure Cyto 180K CGH/SNP arrays (Agilent Technologies, Santa Clara, USA). DNA was labeled (cyanine 3 or cyanine 5) using the Genomic DNA ULS Labeling Kit from Agilent Technologies and hybridized onto the microarrays according to the manufacturer's instructions (Agilent). For SNP+ CGH arrays, tested DNAs were hybridized against male control DNA obtained from Agilent. Microarrays were scanned with a G2565CA scanner (Agilent). Data analysis was carried out with Agilent Technologies software, namely Feature Extraction for Cytogenomics Algorithm V5.0.1.16 to calculate the fluorescence ratio and Agilent CytoGenomics 4.0 and 5.0 to visualize chromosomal imbalances and LOH. Deletions and duplications in the heterozygous state were characterized by values of the log2 ratio of fluorescence intensities (cyanine 5/cyanine 3) below −0.5 and above +0.3, respectively, with the statistical algorithm ADM2 used at a threshold of 5. LOH was evaluated with the statistical algorithm ADM2 used at a threshold of 6 (Default Analysis Method v2).

### Statistics

Statistical significance was inferred when necessary. Exact distinct and independent sample size is indicated in each legend (n). Graph Pad Prism 6 software was used for statistical analysis. Results are presented as mean ±SD. The parametric T-test was used when distribution was Gaussian/normal (Shapiro-Wilk test). The non-parametric Mann-Whitney test (two-sided) was used to compare two groups. One-way ANOVA, complemented with the unprotected Fisher's Least Significant Difference test, was used to compare more than two groups. Percentages of LOH in HSPC were compared by the Chi-square test. Limit of detection of murine FAMReD (mFAMReD), FIG. 7c, was calculated with formulation mean +3SD.

### Results

Detection of megabase-scale rearrangements in human cells: the hFAMReD system. We developed a sensitive approach to detect and quantify LOHs induced by nucleases on human Chr10q and to sort live edited cells for their in-depth characterization. Briefly, hFAMReD relies on a cell phenotype switch, from non-fluorescent to fluorescent, induced by a megabase-scale LOH (data not shown). This switch is due to the accumulation of fluorescent red porphyrins occurring in UROS-deficient cells²², which is readily detectable by flow cytometry upon ALA (5-amino-levulinic acid) precursor exposure (data not shown).

To create this system, immortalized human foreskin fibroblasts (hFFs) were first edited with an ribonucleoprotein (RNP) Cas9: gRNA complex targeting UROS exon 4 in Chr10q26.2 to generate a cell line heterozygous for a loss-of function variant in the UROS gene (UROS^{+/-}) located 7.5 Mb from the telomere (data not shown). Editing efficiency was high (98% of InDels), resulting in 86.2% of fluorescent cells. To select the UROS-hFF clone (with preserved normal heme biosynthesis), we sorted and subcloned the non-fluorescent fraction (data not shown). We selected and amplified one UROS^{+/-} hFF clone and used it thereafter (data not shown).

In this study, we targeted, with the Cas9 nuclease, loci centromeric to UROS, located at least one megabase from UROS and 8.5 Mb from the telomere (data not shown). By doing so, induced megabasic LOHs (copy-loss or copy-neutral) can disrupt the remaining functional UROS allele. The fluorescent switch is then activated without affecting their proliferation (data not shown), and the persistent rearranged LOH⁺ cells can be detected, quantified and sorted for in-depth analyses (data not shown).

### LOH Detection in Fibroblasts (hFFs)

In our previous study, FISH did not reveal a significant level of megabasic CL-LOH in edited WT (p53-proficient) hFFs¹¹. However, FISH sensitivity may not be sufficient to detect rare deletions. Here, we targeted the same chromosome arm (Chr10q) and used hFAMReD to determine whether LOH occurred at low frequency in hFFs. Targeting ABRAXAS2 (10q26.13), 1 Mb centromeric to UROS (data not shown), we observed a slight increase in the level of fluorescent cells (PE-CyA5⁺: 0.09±0.05%) 15 days after editing, suggesting the occurrence of LOH at the UROS locus. Similar results were obtained using the high fidelity HiFi-Cas9nuclease (0.10±0.02%), which was previously reported to have a reduced off-target activity²³. None of the Top-10 predicted off-target sites are located on Chr10q (data not shown), disqualifying an off-target confounding factor as a source of unwanted editing. In contrast, fluorescent cells were absent without transfection and when PIGA, localized on ChrX, was targeted (0.01±0.01% and 0.02±0.01%, respectively (data not shown). These data show that CRISPR-Cas9 nuclease induces megabase-scale LOHs in p53-proficient fibroblasts (at a rate of around one cell with extra-large rearrangement per 1000 edited cells), which is undetectable by FISH. Additionally, no significant increase in fluorescent cell numbers was evidenced using the catalytically inactive deadCas. Using the Cas9^{D10A} nickase, the LOH rate was not significantly different from controls (data not shown). We cannot exclude the possibility of rare LOH with single-strand breaks (SSB) induced by nickase (as observed in ref 11).

We next sorted the fluorescent cells (data not shown) and genotyped UROS. Only the mutated UROS allele was detected, suggesting that all fluorescent cells had lost the UROS wild-type (UROS^{wt}) allele. Conversely, in the non-fluorescent fraction, cells exhibited a UROS^{+/-} heterozygous status comparable to that observed in the initial cell line.

To better delineate and map the DSB-induced LOH, we carried out an array comparative genomic hybridization (cGH) on the sorted fluorescent cell fraction after ABRAXAS2 targeting (data not shown). We detected an interstitial 72 Mb duplication with the distal breakpoint located at the cut-site, and a Log2ratio of +0.3 in favor of a mosaicism, i.e. about 46% of cells carrying interstitial genomic duplication. We also observed an 8.5 Mb terminal deletion with the breakpoint at the cut-site and a Log2 ratio of −0.28 in favor of a mosaicism with 36% of the cells harboring terminal deletion, suggesting the predominant presence of CN-LOH in addition to CL-LOHs. The analysis of two fluorescent clones confirmed the presence of both types of LOH (data not shown): clone #1 presented a terminal CL-LOH starting from the cut-site while clone #2 had a CN-LOH starting from the cut-site, with an extra-large interstitial 10q duplication detected in the bulk analysis. Notably, these genomic abnormalities were not detectable by cGH or by single nucleotide polymorphism (SNP) array without cell sorting (without hFAMRed LOH enrichment, data not shown).

Collectively, these results suggest the occurrence of DSB-induced megabase-scale LOHs in edited hFFs and confirm the high sensitivity of the hFAMReD system to detect and isolate them for precise characterization (data not shown). Importantly, LOH⁺ cells sorted after day 15 were long-term rearranged cells. They persisted at least 40 days, without selective advantage or disadvantage (data not shown).

We then focused on SORCS1, 19 Mb centromeric to UROS for further exploration. For this targeted locus, we obtained a similar level of fluorescent cells (0.09%±0.02, data not shown) compared to the ABRAXAS2-edited hFFs. Again, the sorted fluorescent cells only displayed the UROS loss-of function variant allele (data not shown). Array cGH analysis on SORCSI-edited fluorescent cells did not reveal an abnormality of copy number variation (CNV), in favor of a loss of UROS function by CN-LOH (data not shown). To confirm the presence of extra-large CN-LOH, we identified analyzed SNPs in DOCK1 and MGMT, both located between UROS and the telomere. While the parental cell line was heterozygous for the tested SNPs, 6/6 fluorescent screened clones were homozygous for these SNPs (data not shown), in favor of a CN-LOH from SORCSI to the telomere. A combined array cGH/SNP array in the same clones demonstrated the absence of deletion and mapped a 26.5 Mb terminal CN-LOH, starting from the cut-site (data not shown).

To evaluate whether the location of DSB affects the frequency and the LOH type, we next targeted hChr10 by five other gRNA targeting CPXM2, PLEKHA1, TRUB1, ADRA2A and VCL respectively 2, 4, 10, 14 and 50 megabases centromeric from UROS (data not shown). For each guide RNA, we obtained a high InDel rate (>50%) and very similar LOH frequencies (from 0.1% to 0.2% of fluorescent cells in WT hFFs, even with the farthest away). The Pearson correlation coefficient was low (R²=0.22), demonstrating the absence of correlation between the distance to the telomere and the LOH frequency (data not shown). Interestingly, cGH-arrays of the fluorescent cell bulk for each additive gRNA were normal (data not shown), suggesting that CN-LOH are predominant. When we targeted loci more than 2 Mb away from UROS, CL-LOH were under the cGH-array limit of detection (10%).

Altogether, these results seem to indicate that the targeted locus does not influence the frequency of the total LOH rate but can modify the relative proportion of CN-LOH and CL-LOH.

### p53 Inhibition Increases DSB-Induced LOH Frequency

p53 plays an essential role in DNA damage sensing and repair^{24,25} and is activated by CRISPR-induced DSB²⁶⁻²⁹. We previously demonstrated the strong involvement of p53 in chromosomal instability induced by CRISPR-Cas9 with a dramatic increase in megabase-scale CL-LOH in p53-deficient hFFs¹¹. However, the impact of p53 deficiency on the global LOH rate, including CN-LOH and CL-LOH, is still unknown. TP53 was invalidated with an RNP Cas9: gRNA targeting TP53 in the hFAMReD UROS^{+/-} hFFs, using the same RNP previously reported¹¹. We selected a clone homozygous for a loss of function variant in TP53 (TP^{-/-}, data not shown), with a normal karyotype (data not shown). In p53^{-/-} fibroblasts, ABRAXAS2 and SORCS1 targeting (located 1 Mb and 19 Mb centromeric to UROS, respectively) led to the occurrence of 5.6%±0.8 and 5.56%±0.3 of fluorescent cells, respectively (data not shown), a 60-fold increase compared to p53-proficient-hFFs. Minimal levels of fluorescent cells were observed in non-transfected cells and in cells edited with an RNP targeting PIGA (located on ChrX) (data not shown). Again, HiFi-Cas9 editing did not decrease the rate of fluorescent cells (6.2±0.6%, data not shown) and LOH frequencies were independent of distance-to-telomere and of OFF-target events.

After editing of either ABRAXAS2 or SORCS1, sequencing of fluorescent cells confirmed the loss of the UROSWT allele (data not shown), and SNP allelic losses for DOCK1 and MGMT for ABRAXAS2 (data not shown), indicating that the fluorescent cells harbored LOHs encompassing UROS and genes near to the telomere. Array cGH of polyclonal fluorescent ABRAXAS2-edited cells revealed a mosaic Chr10q 26.13qter deletion (Log2ratio:-0.33, 41% of cells) in favor of a mix of CL-LOH and CN-LOH (data not shown). In contrast, array cGH was almost normal after SORCS1 targeting, in favor of rare deletions (Log2ratio-0.08, 10% of alleles) and a high prevalence of CN-LOH (data not shown).

Taken together, these results confirm the role of p53 in maintaining genome stability after a CRISPR-Cas9 DSB. They precisely quantify the megabase-scale LOH increased risk in p53-deficient cells compared to p53-proficient cells.

### Cell Cycle Drives DSB-Induced Megabase-Scale ON-Target LOHs.

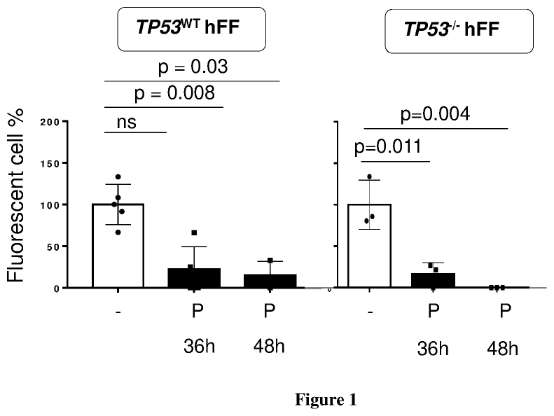

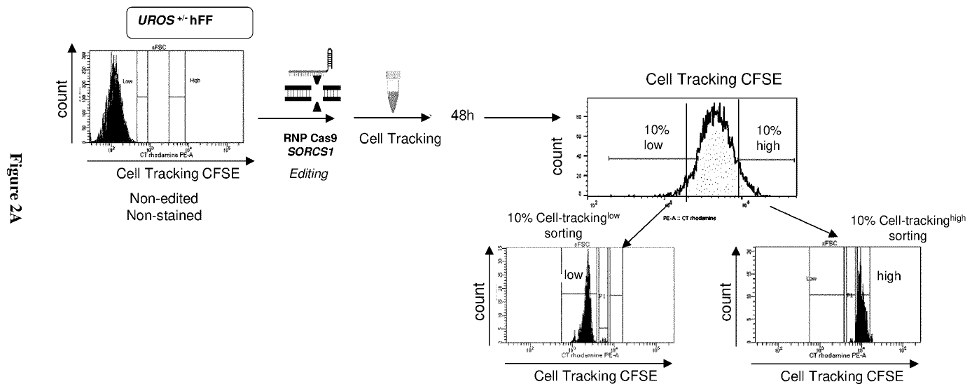

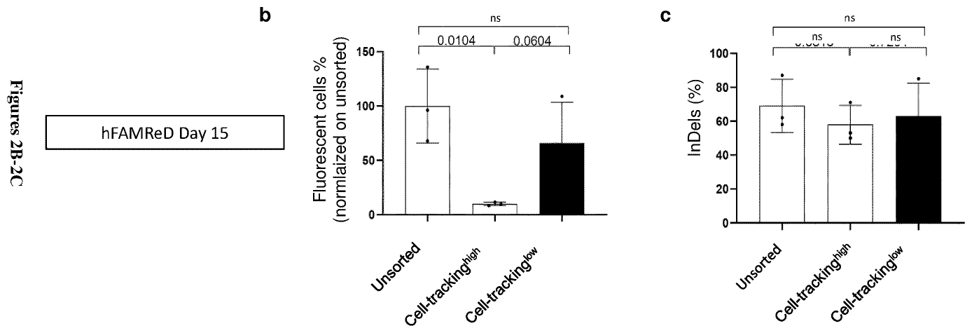

p53 plays a critical role in both the G1/S and G2/M cell cycle checkpoints³¹. In response to DNA DSBs, cell cycle is arrested by activation of these cell-cycle checkpoints to facilitate DSB repair by non-homologous end-joining (NHEJ) or homologous recombination (HR). Conversely, a DSB during replication(S) and mitosis (M) could be deleterious because it can trigger replication fork collapse and induce missegregation of acentric fragments³². We hypothesized that the cell cycle phase during which the DSB occurs could influence the LOH rate. To test this hypothesis, we edited synchronized p53-proficient cells in G0/G1 and G2/M phases using palbociclib³³, a CDK4/6 inhibitor and RO-330634, a selective CDK1 inhibitor, respectively (FIG. 1). Exposure was maintained 24 h or 48 h after transfection to cover the Cas9 RNP activity window. RO-3306 induced a partial cell entry in the M phase and did not modify LOH frequency (FIG. 1). In contrast, with palbociclib, which blocks cells in G0/G1 during editing (data not shown), LOH frequencies were dramatically reduced (FIG. 1) in p53-proficient and p53-deficient cells. Notably, editing efficiency at SORCS1 was maintained similar to non-synchronized cells. Therefore, our data demonstrate that the cell cycle plays a key role in genotoxicity induced by DSB. Editing during G0/G1 limits megabase-scale LOHs, even in p53-invalidated cells. Therefore, it is possible to prevent LOH by controlling cell cycle.

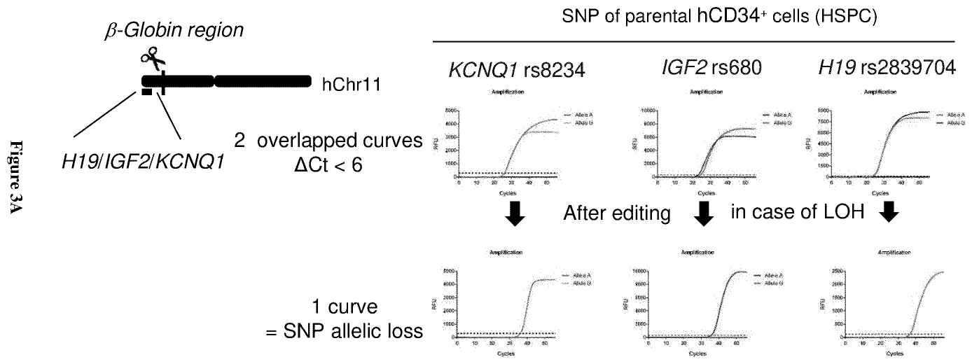

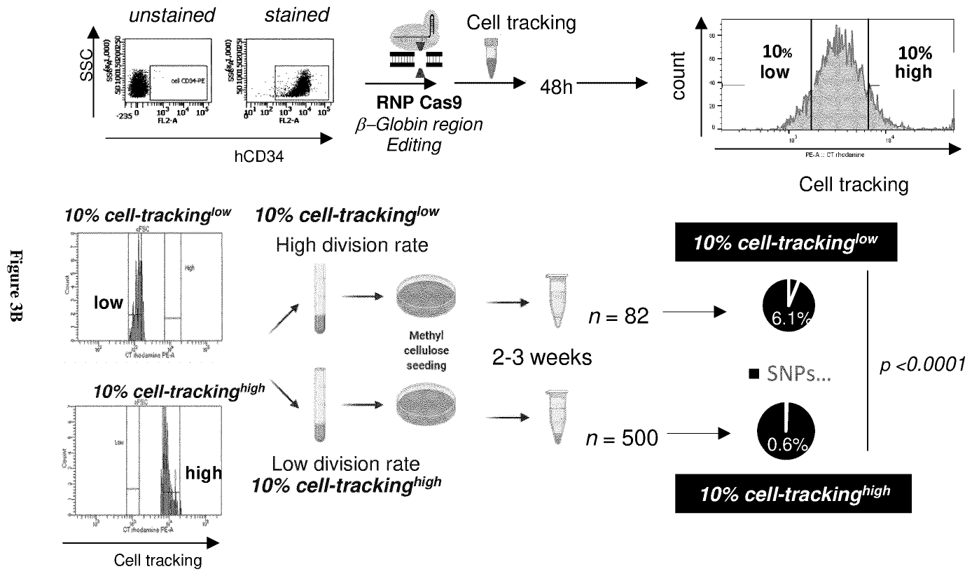

To confirm the hypothesis that cell division rate can impact LOH frequency, we stained hFAMReD hFFs just before editing with fluorescent cell tracking to monitor cell generations by dilutions. Two days post-editing, we sorted the 10% of cells with the highest mean fluorescence intensity (MFI), hereafter referred as cell-tracking^{high} (FIG. 2). This fraction corresponds to the cells with a ‘low division rate’, confirmed by a higher BrDU^{neg} fraction (data not shown). The 10% of cells with the lowest MFI (cell-tracking^{low}) correspond to the cells with the most active proliferation. Editing efficiency was similar in the three cell fractions (around 60% of InDels). At day 15 post-editing, cell-tracking^{high} fraction had a dramatically reduced LOH rate compared to unsorted (21.7-fold decrease) and to cell-tracking^{low} cells (27.4-fold decrease, FIG. 2), without impairing InDel frequency.

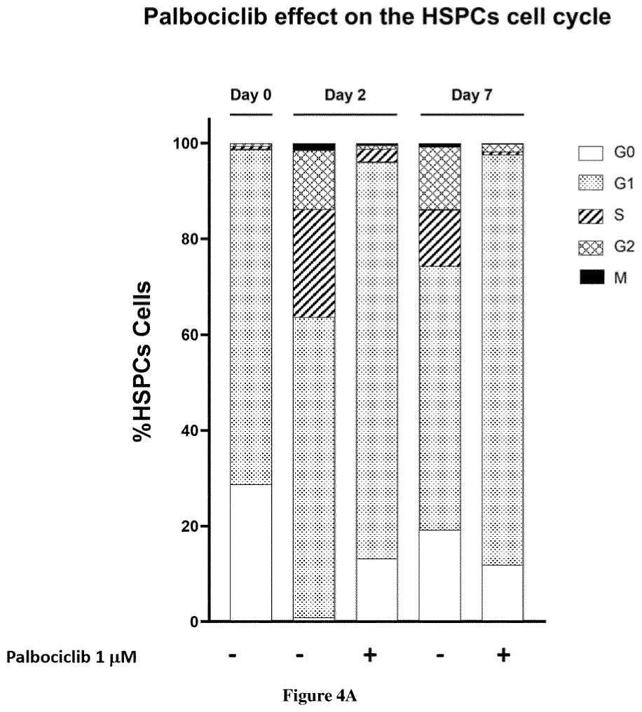

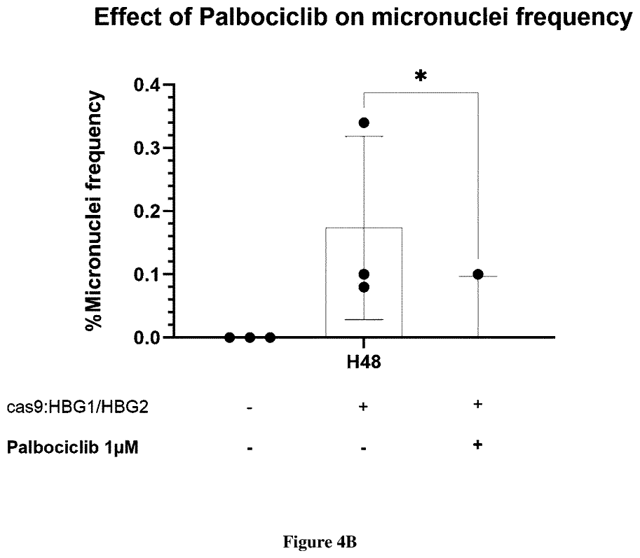

To confirm that cell division control can prevent megabase-scale DSB-induced LOH, we targeted the clinically relevant globin cluster on Chr11p15.4 (FIG. 3) in HSPCs. We previously reported around 1% of LOH after editing this locus¹⁵. Using the same protocol as described above, we analyzed LOH frequency by SNP allelic losses in HSPC subfractions sorted according to their proliferation activity. To do so, we first identified informative SNPs in the H19/IGF2/KCNQ1 imprinting center 2.5 Mb telomeric to the cut-site (heterozygous SNP in parental cells). We considered that allelic loss in at least two telomeric SNPs was necessary to establish the existence of a telomeric megabase-scale LOH. We then sorted cell-trackinghigh and cell-trackinglow edited HSPCs and performed a colony-forming cell (CFC) assay to monitor SNP genotypes at a clonal level. Despite high InDel frequency (93%) in cell-trackinghigh cells, single cell SNP analysis revealed a lower rate of LOH (0.6%, n=500 clones) than in cell-trackinglow HSPCs (6.1%, n=82 clones) (FIG. 3).

Taken together, hFAMReD showed that megabase-LOH genotoxicity is cell-division-dependent. Importantly, we confirmed this new concept in clinically relevant cells for gene therapy protocols using CRISPR-Cas9 nuclease.

### Palbociclib Prevent the Formation of Micronuclei (Small DNA-Containing Structures Isolated From the Main Nucleus)

We then study the effect of Palbociclib on the formation of micronuclei (small DNA-containing structures isolated from the main nucleus). These micronuclei evokes a massive DNA rearrangement called chromothripsis. We demonstrate that Palbociclib prevent the formation of micronuclei as depicted in FIGS. 4A and 4B.

### Discussion

Successful detection, quantification and characterization of unwanted OFF-and ON-target events after editing by nucleases are crucial for safe gene therapy. Megabase-scale ON-target genotoxicity was first described in cell lines^{11,16}, and more recently in primary cells^{9,13,16,17,21}, targeting different loci, with highly variable frequencies. It is unclear whether the choice of repair pathway or fails in repairing the breaks are dependent on the chromosomal DSB location and cell properties, and whether these genomic rearrangements are stable overtime. In this study, we developed a FAMReD system, a new method to study unintended terminal megabase-scale by-products induced by CRISPR-Cas9, targeting hChr10. hFAMReD is able to detect the appearance of persistent events by red fluorescence and to quantify them (only after day 15) but with high sensitivity (0.02%) in a cell bulk. With a limit of detection around 5-10%, FISH, array CGH, and SNP array (without subcloning/single cell analysis) all lack sensitivity to detect these rare rearrangements present in p53-proficient cells.

Although very efficient to enrich cells for LOH rearrangements, FAMReD is limited to hChr10q genotoxicity analysis, so DSBs on other chromosomes cannot be explored. Alternatively, targeted installation of two fluorescent tags on homologous chromosome telomeres, which are lost with megabase-scale LOH, could be another quality control system to assess genotoxicity in clinical trials. Nevertheless, inversions, balanced translocations, and rearrangements centromeric to the cut-site would not be detectable. Recently, single-cell RNAseq was proposed to sensitively detect copy-loss LOH²¹. However, preponderant copy-neutral LOH was not detected and rearranged cells could not be sorted for characterization. This highlights the need for combinatorial methods for genotoxicity exploration at the nucleotide (e.g. NGS, long-range PCR) and chromosome levels (e.g. CAST-seq, FAMReDs, single-cell SNP analysis) to precisely determine the genotype of edited cells.

Even if both FAMReD systems are not applicable for testing clinically relevant CRIPSR-Cas9 targets, they highlight general mechanisms to understand and prevent genotoxicity. In this study, using hFAMReD, we observed that the frequencies of fluorescent cells were similar when targeting many loci located from 8.5 to 57.5 Mb from the telomere, respectively, suggesting that the LOH rate does not depend on the distance to the telomere. Importantly, regardless of the distance to the telomere, CN-LOH are predominant. CL-LOH were observed only with the most telomeric cut. We speculate that the real LOH frequency can be obtained by doubling the observed fluorescent cell percentages (hFAMReD only detect LOHs by UROS WT).

Importantly, LOH were quantified by hFAMReD from 2 weeks post-editing, suggesting that the observed genotoxicity is persistent and not eliminated shortly after editing by p53 activation and cell death.

Invalidation of TP53 led to a dramatic increase in LOH levels, further confirming that it is a key factor ensuring proper DNA damage response and genome stability²⁸. Transient p53 inhibition using two inhibitors and targeting two loci moderately increased LOH frequency. It appears that enhancing gene editing efficiency by modulating p53 activity should be carried out with careful monitoring of genome integrity post-editing. p53 is a key regulator of cell cycle checkpoint and division³⁵⁻³⁷. Good timing in the cell cycle is known to be important to understand and improve editing. While NHEJ is known to be active throughout the cell cycle, HR is the most active during the S phase. Here, we highlight the impact of the cell division rate on genotoxicity during editing in hFFs and HSPCs. G0/G1 synchronized cells and low-divided cells were protected from LOHs. Cells may be protected by enhanced canonical-NHEJ^{38,39} Indeed, the c-NHEJ repair pathway could limit LOH by joining both chromosome parts after DSB. This would be in accordance with two studies^{40,41} showing that when cNHEJ is deficient, DSBs are repaired by genotoxic pathways and induce kilobasic rearrangements. Here, we demonstrate that a low proliferation rate strongly reduces the occurrence of LOHs without compromising NHEJ editing efficiency (in two human cell models, targeting two different chromosomes and using two methods to quantify LOH). The relative risk of megabase-scale LOHs induced by CRISPR is reduced 10-fold in hCD34⁺ cells with a low division rate. Importantly, cell cycle arrest by palbociclib was able to suppress genotoxicity even in p53-deficient cells. This novel concept should be considered when designing clinical cell culture protocols before editing. Slowing down cell proliferation before editing could decrease this ON-target genotoxicity.

By leveraging the phenotype switch, FAMReD offer another advantage in the possibility of isolating cells with stable LOHs for in-depth analysis. Here, using hFAMReD, we isolated rearranged cells and observed different types of LOH, (CL-LOH or CN-LOH), starting from the cut-site to the telomere. Interestingly, the rearrangement type profile (CN-LOH/CL-LOH ratio) was distinct when targeting different loci. In all cases, CN-LOH were predominant. We only observed CL-LOH in the event of DSB at the closest target to the telomere (8.5 Mb). Notably, the rearrangement type profile was not modified by TP53 invalidation for a defined locus. Further studies will be required to evaluate whether CN-LOHs, which are not detectable by FISH or array CGH, have a functional impact, depending on the genes in the chromosomal region of interest. These CN-LOH are probably due to a loss of chromosome extremity and to a secondary duplication of the remaining allele by break-induced replication (BIR)⁴⁵ to avoid CNV. These large LOHs could contribute to tumorigenesis by activating potential oncogenes or by unmasking mutated tumor suppressor genes.

In this study, LOH was sometimes associated with centromeric extra-large duplication, again illustrating the diversity of ON-target rearrangements induced by CRISPR-Cas9 nuclease. Complex large kilobase-scale rearrangements have already been reported^{6,10}. The mechanisms of this unexpected rearrangement linking duplication with terminal LOH remain elusive. It could be due to a U-type exchange, a breakage-fusion-bridge process⁴⁶, non-allelic homologous recombination, a fold-back mechanism⁴⁷, or possibly chromothripsis^{17,48}.

In conclusion, we have developed a new cytometry-based megabase-scale LOH detection systems based (FAMReD). It revealed and quantified the genotoxicity of CRISPR-Cas9 in primary cells with high sensitivity. Importantly, we identified mechanisms linking p53, cell division and genotoxicity. Cell cycle blockade by palbociclib prevents ON-target megabase-scale genotoxicity without compromising the efficacy of NHEJ. These data offer new opportunities to make nuclease-based gene therapy protocols safer.

### REFERENCES

Throughout this application, various references describe the state of the art to which this invention pertains. The disclosures of these references are hereby incorporated by reference into the present disclosure.
1. Rouet, P., Smih, F. & Jasin, M. Expression of a site-specific endonuclease stimulates homologous recombination in mammalian cells. Proc. Natl Acad. Sci. USA 91, 6064-6068 (1994).
2. Jasin, M. Haber, J. E. The democratization of gene editing: Insights from site-specific cleavage and double-strand break repair. DNA Repair (Amst). 44, 6-16 (2016).
3. Doudna, J. A. & Charpentier, E. Genome editing. The new frontier of genome engineering with CRISPR-Cas9. Science 346, 1258096 (2014).
4. Cong, L. et al. Multiplex genome engineering using CRISPR/Cas systems. Science 339, 819-823 (2013).
5. Boutin, J. et al. ON-Target Adverse Events of CRISPR-Cas 9 Nuclease: More Chaotic than Expected. CRISPR J. 5, 19-30. (2022).
6. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
7. Shin, H. Y. et al. CRISPR/Cas9 targeting events cause complex deletions and insertions at 17 sites in the mouse genome. Nat. Commun. 8, 15464(2017 ).
8. Rezza, A. et al. Unexpected genomic rearrangements at targeted loci associated with CRISPR/Cas9-mediated knock-in. Sci Rep. 9, 3486(2019 ).
9. Weisheit, I. et al. Detection of deleterious on-target effects after HDR mediated CRISPR editing. Cell Rep. 26, 107689(2020 ).
10. Alanis-Lobato, G. et al. Frequent loss of heterozygosity in CRISPR-Cas9-edited early human embryos. Proc. Natl Acad. Sci. USA 118, e2004832117 (2021).
11. Cullot, G. et al. CRISPR-Cas9 genome editing induces megabase-scale chromosomal truncations. Nat. Commun. 10, 1136(2019 ).
11. Rayner, E. et al. CRISPR-Cas9 Causes Chromosomal Instability and Rearrangements in Cancer Cell Lines, Detectable by Cytogenetic Methods. CRISPR J. 2, 406-416 (2019).
13. Zuccaro, M. V. et al. Allele-specific chromosome removal after Cas9 cleavage in human embryos. Cell 29, 31389-31391 (2020).
14. Davis, L. et al. POLQ suppresses interhomolog recombination and loss of heterozygosity at targeted DNA breaks. Proc. Natl Acad. Sci. USA 15, 22900-22909 (2020).
15. Ikeda, K. et al. Efficient scarless genome editing in human pluripotent stem cells. Nat Methods. 15, 1045-1047 (2018).
16. Boutin, J. et al. CRISPR-Cas9 globin editing can induce megabase-scale copy-neutral losses of heterozygosity in hematopoietic cells. Nat Commun. 12, 4922(2021 ).
17. Leibowitz, M. L. et al. Chromothripsis as an on-target consequence of CRISPR-Cas9 genome editing. Nat. Genet. 53, 895-905 (2021).
18. Papathanasiou, S. et al. Whole chromosome loss and genomic instability in mouse embryos after CRISPR-Cas9 genome editing. Nat. Commun. 12, 5855(2021 ).
19. Zuo, E. et al. CRISPR/Cas9-mediated targeted chromosome elimination. Genome Biol. 18, 224(2017 ).
20. Adikusuma, F. et al. Large deletions induced by Cas9 cleavage. Nature 560, E8-E9 (2018).
21. Nahmad, A.D. et al. Frequent aneuploidy in primary human T cells after CRISPR-Cas9cleavage. Nat Biotechnol. 12,1807-1813 (2022).
22. Puy, H. Gouya, L. Deybach, J. C. Porphyrias. Lancet. 375, 924-937 (2010).
23. Vakulskas, C. A. Behlke, M. A. Evaluation and Reduction of CRISPR Off-Target Cleavage Events. Nucleic Acid Ther. 29, 167-174 (2019).
24. Vousden, K. H. Lane, D. P. p53 in health and disease. Nat Rev Mol Cell Biol. 8, 275-283 (2007).
25. Levine, A. J. p 53: 800 million years of evolution and 40 years of discovery. Nat Rev Cancer 20, 471-480 (2020).
26. Ihry, R. et al. p53 inhibits CRISPR-Cas9 engineering in human pluripotent stem cells. Nat. Med. 24, 939-946 (2018).
27. Haapaniemi, E. et al. CRISPR-Cas9 genome editing induces a p53-mediated DNA damage response. Nat. Med. 24, 927-930 (2018).
28. Jiang, L. CRISPR/Cas9-Induced DNA Damage Enriches for Mutations in a p53-Linked Interactome: Implications for CRISPR-Based Therapies. Cancer Res. 82, 36-45 (2022).
29. Enache, O. M. et al. Cas9 activates the p53 pathway and selects for p53-inactivating mutations. Nat Genet. 52, 662-668 (2020).
30. Schiroli, G. et al. Precise Gene Editing Preserves Hematopoietic Stem Cell Function following Transient p53-Mediated DNA Damage Response. Cell Stem Cell. 24, 551-565 (2019)
31. Schwartz D, Rotter V. p53-dependent cell cycle control: response to genotoxic stress. Semin Cancer Biol.8, 325-336 (1998).
32. Levine, M. S. ; Holland, A. J. The impact of mitotic errors on cell proliferation and tumorigenesis. Genes Dev.32, 620-638 (2018).

33. Schmidt M. Palbociclib—from Bench to Bedside and Beyond. Breast Care. 11, 177-181 (2016).
34. Vassilev L T, et al. Selective small-molecule inhibitor reveals critical mitotic functions of human CDK1. Proc Natl Acad Sci U S A. 103, 10660-10665 (2006).
35. Halazonetis T D, Gorgoulis V G, Bartek J. An oncogene-induced DNA damage model for cancer development. Science. 319, 1352-1355 (2008).
36. Roy, S. et al. p53 orchestrates DNA replication restart homeostasis by suppressing mutagenic RAD52 and POLθ pathways. Elife. 7, e31723 (2018).
37. Kondratova, A. Replication fork integrity and intra-S phase checkpoint suppress gene amplification. Nucleic Acids Res. 43, 2678-2690 (2015).
38. Her, J. Bunting, S. F. How cells ensure correct repair of DNA double-strand breaks. J Biol Chem. 293, 10502-10511 (2018).
39. Pesch, A. M. et al. Short-term CDK4/6 Inhibition Radiosensitizes Estrogen Receptor-Positive Breast Cancers. Clin Cancer Res. 26, 6568-6580 (2020).
40. Kosicki, M. et al. Cas9-induced large deletions and small InDels are controlled in a convergent fashion. Nat Commun. 13, 3422(2022 ).
41. Arlt, M. F. Replication stress induces genome-wide copy number changes in human cells that resemble polymorphic and pathogenic variants. Am J Hum Genet. 84, 339-350 (2009).
42. Anzalone, A. V., Koblan, L. W. & Liu, D. R. Genome editing with CRISPR-Cas nucleases, base editors, transposases and prime editors. Nat. Biotechnol. 38, 824-844 (2020).
43. Rees, H. A. & Liu, D. R. Base editing: precision chemistry on the genome and transcriptome of living cells. Nat. Rev. Genet. 19, 770-788 (2018).
44. Tran, N. T. Precise CRISPR-Cas-mediated gene repair with minimal off-target and unintended on-target mutations in human hematopoietic stem cells. Sci Adv. 8, eabm9106 (2022).
45. Kramara, J., Osia, B. & Malkova, A. Break-induced replication: the where, the why, and the how. Trends Genet. 34, 518-531 (2018).
46. Murnane, J. P. Telomere dysfunction and chromosome instability. Mutat Res. 730, 28-36 (2012).
47. Hermetz, K. E. et al. Large inverted duplications in the human genome form via a fold-back mechanism. PLOS Genet. 10, e1004139 (2014).
48. Maciejowski, J. et al. Chromothripsis and Kataegis Induced by Telomere Crisis. Cell. 163, 1641-1654 (2015).
49. Zhou B.P. et al. HER-2/neu induces p53 ubiquitination via Akt-mediated MDM2 phosphorylation. Nat. Cell. Biol. 11, 973-982 (2001).
50. Zhou B. P. et al. Cytoplasmic localization of p21Cip1/WAF1 by Akt-induced phosphorylation in HER-2/neu-overexpressing cells. Nat Cell Biol. 3, 245-252 (2001).

1. A method of preventing ON-target genotoxicity induced by a nuclease in a population of cells comprising the steps of i) inducing G0/G1 phase cell cycle arrest in the population of cells by contacting the cells with an agent that selectively induces G1 phase cell cycle arrest and ii) editing the population of cells induced in step i) with the nuclease.

2. The method of claim 1, wherein the method reduces loss of heterozygosity (LOH) frequency.

3. The method of claim 2, wherein the method reduces nuclease-induced megabase-scale LOH.

4. The method according to claim 1, wherein the nuclease is a TALEN, a Zinc-Finger Nuclease (ZFN) or a a CRISPR/Cas nuclease.

5. The method of claim 4 wherein the CRISPR/Cas nuclease is Cas9 nuclease.

6. A method of altering a target sequence of a nucleic acid molecule in a population of cells for which a G0/G1 phase cell cycle arrest is induced comprising contacting the target sequence of the nucleic acid molecule with (a) a nuclease and (b) one or more guide RNA(s).

7. The method according to claim 1, wherein the population of cells is a population of eukaryotic cells.

8. The method of claim 7 wherein the eukaryotic cells are selected from the group consisting of hematopoietic progenitor cells, hematopoietic stem cells (HSCs), pluripotent cells and induced pluripotent stem cells (iPS)).

9. The method according to claim 1, wherein the cells are p53-proficient cells or p53-deficient cells.

10. The method according to claim 1, wherein the agent that selectively induces G1 phase cell cycle arrest is a selective CDK4/6 inhibitor.

11. The method of claim 10 wherein the selective CDK4/6 inhibitor is selected from the group consisting of abemaciclib, palbociclib, and ribociclib.

12. The method of claim 11 wherein the selective CDK4/6 inhibitor is Palbociclib.

13. The method according to claim 1, wherein the steps of inducing and editing occurs simultaneously.

14. A method of treating or preventing a disorder associated with increased expression of a target polynucleotide sequence in a patient in need thereof, comprising transplanting into the patient a therapeutically effective amount of a population of edited eukaryotic cells in which the target polynucleotide sequence is edited by the method according to claim 1.

15. A method of treating or preventing a disorder associated with increased expression of a target polynucleotide sequence in a patient in need thereof, comprising administering to the patient a therapeutically effective amount of a nuclease in combination with a therapeutically effective amount of an agent that induces G0/G1 cell cycle arrest.

16. The method of claim 3, wherein the megabase-scale LOH is megabase-scale loss-of-heterozygosity with copy losses (CL-LOH) and/or megabase-scale copy-neutral loss-of-heterozygosity (CN-LOH).

17. The method of claim 8, wherein the pluripotent cells are embryonic stem cells (ES).
